(19) United States
(12) Patent Application Publication
(10) Pub. No.: US 2025/0186236 A1
(43) Pub. Date: Jun. 12, 2025

(54) BRACING DEVICE FOR RESTORING NATURAL FOOT MOVEMENT TO FLAT FOOT DEFORMITY PATIENTS

(71) Applicant: ARIZONA BOARD OF REGENTS ON BEHALF OF THE UNIVERSITY OF ARIZONA, Tucson, AZ (US)

(72) Inventors: L. Daniel Latt, Tucson, AZ (US); Aaron Russel Ramos, Tucson, AZ (US); Vinamra Kumar, Tucson, AZ (US); MacKenzie McNeil, Tucson, AZ (US); Sebastian Diaz, Tucson, AZ (US); Alex Joseph Sciortino, Tucson, AZ (US)

(21) Appl. No.: 18/973,590

(22) Filed: Dec. 9, 2024

Related U.S. Application Data
(60) Provisional application No. 63/608,577, filed on Dec. 11, 2023.

(51) Int. Cl. A61F 5/01

(52) U.S. Cl. CPC A61F 5/0127; A61F 2005/0172

(57) ABSTRACT

A dynamic foot brace device for providing arch support to a foot of a user through energy storage at an ankle of the user during a step. The device includes a tension member including a first end coupled to an arch of the foot and a second end coupled to the ankle. The tension member may be configured to store energy from tension applied to the first end during a first phase of the step and release the energy to the foot as a propulsion force when the tension applied to the first end is released during a second phase of the step.

### CROSS-REFERENCES TO RELATED APPLICATIONS

This application is a non-provisional and claims benefit of U.S. Patent Application No. 63/608,577, filed Dec. 11, 2023, the specification of which is incorporated herein in its entirety by reference.

### FIELD OF THE INVENTION

The present invention is directed to a foot brace for aiding users suffering from acquired flat foot deformity while restoring the natural motion of the foot.

### BACKGROUND OF THE INVENTION

Adult-acquired flat foot deformity (AAFD), also known as Posterior Tibial Tendon Dysfunction (PTTD), Posterior Tibial Tendon Insufficiency (PTTI), Progressive Collapsing Foot Deformity (PCFD), or degenerative flatfoot, is a common degenerative condition characterized by the collapse of the arch leading to foot deformity. This condition causes ankle pain, swelling, and limited mobility which inhibits the individual's ability to engage in daily activities. Current solutions for this condition often involve static braces or orthotics that provide limited support and fail to address the dynamic needs of the foot during motion. These conventional approaches lack the ability to restore the natural motion of the foot, causing users to experience a restricted range of movement, discomfort from the brace, and limited improvement in walking ability.

Because of the structure of the transverse tarsal joint (composed of the talonavicular (TN) and calcaneus-cuboid (CC) joints), the foot is rigid when the midfoot is inverted on the hindfoot (varus) and flexible when the midfoot is everted (valgus). The foot moves from inversion to eversion and back to inversion during the gait cycle. During the swing phase and heel strike, the foot is inverted, it then rapidly everts causing the foot to soften and absorb shock. As the body comes over the foot in mid-stance, the combination of the windlass mechanism, the tightening of the plantar fascia from dorsiflexion of the toes, and the action of the PTT cause the foot to invert making it rigid again so that it can be used as a lever during pushoff. Currently available static braces rigidly hold the foot in an inverted position preventing the natural inversion and eversion during the gait cycle. Thus, there exists a present need for a brace capable of restoring the alignment of the foot, while preserving natural foot motion in users suffering from acquired flat foot deformity.

### BRIEF SUMMARY OF THE INVENTION

It is an objective of the present invention to provide devices that allow for a foot brace for aiding users suffering from flat foot deformity while restoring the natural motion of the foot, as specified in the independent claims. Embodiments of the invention are given in the dependent claims. Embodiments of the present invention can be freely combined with each other if they are not mutually exclusive.

This technology is a dynamic brace that incorporates an energy-storing mechanism to address degenerative flat foot, a condition characterized by arch collapse. Unlike static braces, this solution harnesses the power of energy storage to act as a conduit for dynamic motion and propel the wearer forward, effectively transforming each step into a responsive spring-like motion. The technology not only addresses the physical challenges of degenerative flat feet but also returns energy to the user's gait, empowering individuals with a renewed freedom of movement while allowing for storage of the inversion/eversion mechanism of the foot. The brace of the present invention stores energy during the initial stance as the foot goes from inversion to eversion and then returns it to the foot at midstance to assist with inverting the foot to get ready for terminal stance and push-off.

The present invention features a dynamic brace that relies on energy storage and returns motion which helps spring you forward. The spring means can be any cushiony thing such as an elastomeric spring or carbon fiber or kinetic storing, motion, energy capture/return means. The ankle, heel cuff, and arch support communicate with the spring as seen in the attached drawings.

The present invention features a dynamic foot brace device for providing arch support to a foot of a user through energy storage at an ankle of the user during a step. The device may comprise a tension member comprising a first end coupled to an arch of the foot and a second end coupled to the ankle. The tension member may be configured to store energy from tension applied to the first end during a first phase of the step and release the energy to the foot as a propulsion force when the tension applied to the first end is released during a second phase of the step.

One of the unique and inventive technical features of the present invention is the implementation of an energy-storing tension member paired with an ankle/arch cuff. Without wishing to limit the invention to any theory or mechanism, it is believed that the technical feature of the present invention advantageously provides for flat foot deformity relief without restricting the natural movement of the foot by harnessing the natural kinetic energy stored by the tension member for restoring the natural inversion-eversion motion of the foot. None of the presently known prior references or work has the unique inventive technical feature of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows a system block diagram of the dynamic foot brace of the present invention.

FIG. 1B shows a flow chart of a method for providing arch support to a foot of a user through energy storage at an ankle of the user.

FIG. 1C shows a diagram of a first phase of a step, where the user's weight is shifted onto the foot.

FIG. 1D shows a diagram of a second phase of a step, where the user's weight is taken off of the foot and the foot is lifted and brought forward.

FIG. 2A shows a front isometric view of the dynamic foot brace of the present invention.

FIG. 2B shows a back isometric view of the dynamic foot brace of the present invention.

FIG. 2C shows a side profile view of the dynamic foot brace of the present invention.

FIG. 2D shows a top view of the dynamic foot brace of the present invention.

FIG. 3A shows an isometric view of the tension member of the dynamic foot brace of the present invention.

FIG. 3B shows a top view of the tension member of the dynamic foot brace of the present invention.

FIG. 3C shows a side profile view of the dynamic foot brace of the present invention.

FIG. 4A shows an isometric view of the arch support component of the dynamic foot brace of the present invention.

FIG. 4B shows a side profile view of the arch support component of the dynamic foot brace of the present invention.

FIG. 4C shows a front view of the arch support component of the dynamic foot brace of the present invention.

FIG. 5A shows an isometric view of the tension member guide of the dynamic foot brace of the present invention.

FIG. 5B shows a side profile view of the tension member guide of the dynamic foot brace of the present invention.

### DETAILED DESCRIPTION OF THE INVENTION

Following is a list of elements corresponding to a particular element referred to herein:
100 device
110 sleeve component
120 tension member
130 arch support component
140 tension member guide component

The term “energy” is defined herein as a force that is transmitted through pulling an object tightly, trying to restore the object to its original, unstretched length.

The term “storing” is defined herein as maintaining the energy by keeping the object (e.g. the tension member) at a stretched length.

The term “releasing” is defined herein as allowing the object (e.g. the tension member) to return to its original, unstretched length, causing the stored energy to be applied to the object to achieve this return.

The term “propulsion force” is defined herein as a force that causes motion of some kind.

The term “non-collapsed arch” is defined herein as an arch of a foot that has a healthy, normal, lifted shape.

The term “first phase of the step” is defined herein as the stage of a step where the user's weight is shifted onto the foot.

The term “second phase of the step” is defined herein as the stage of a step the user lifts their foot from the ground and brings it forward.

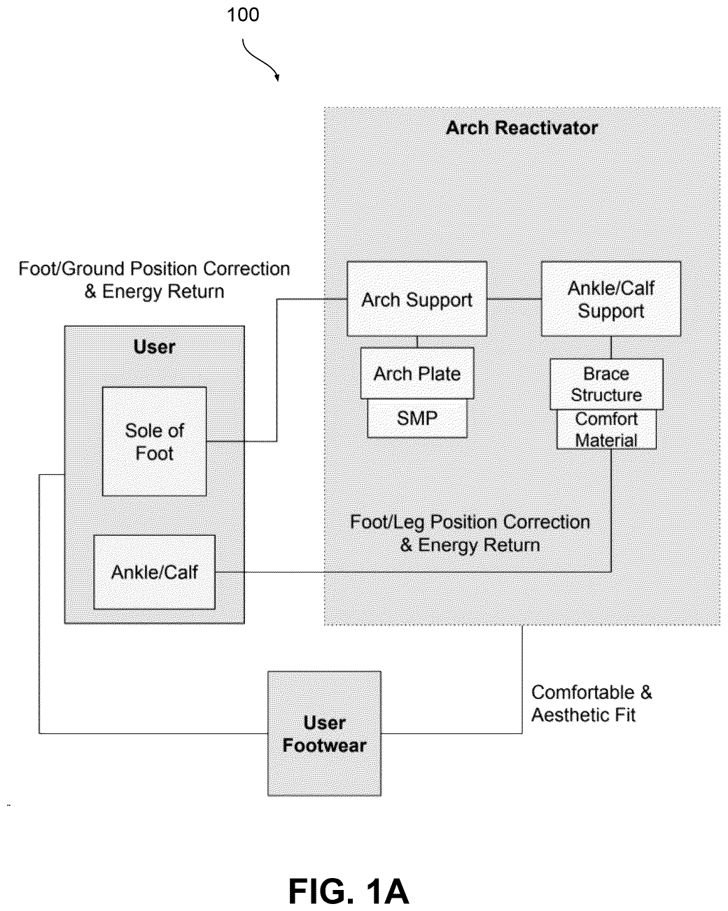

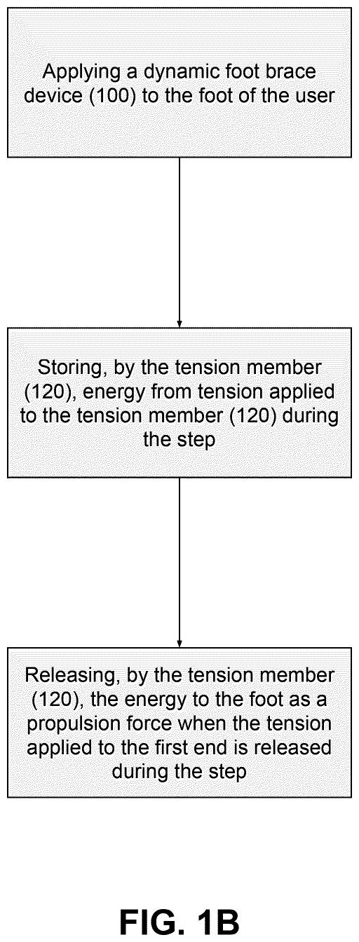

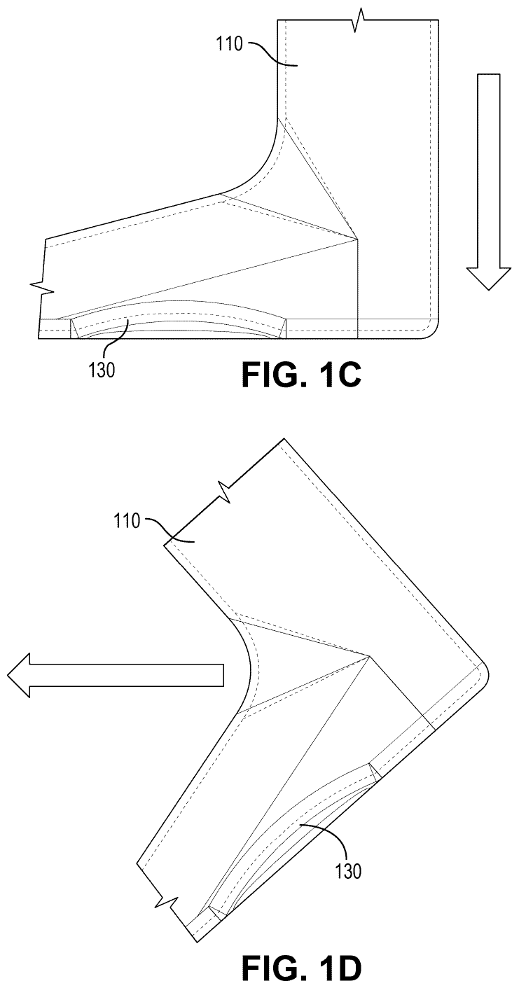

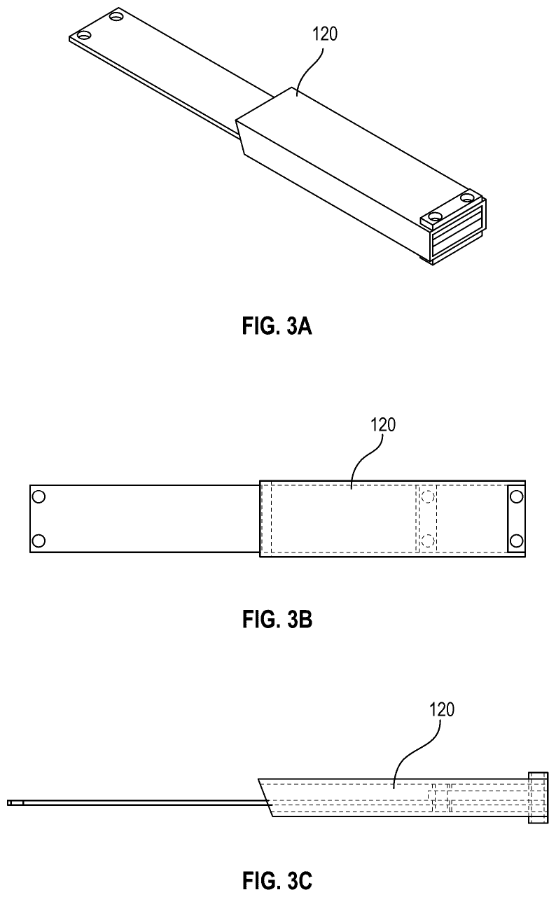

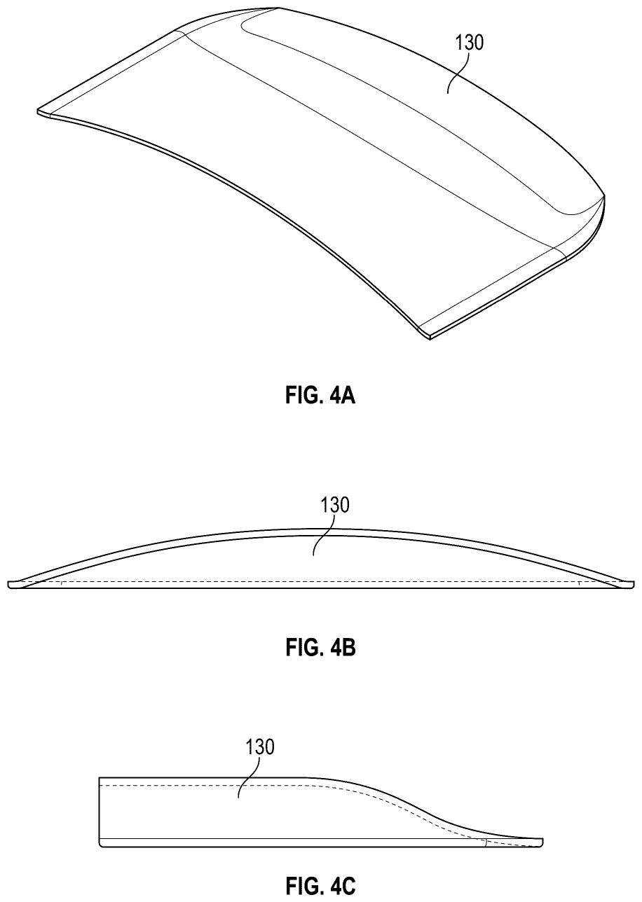

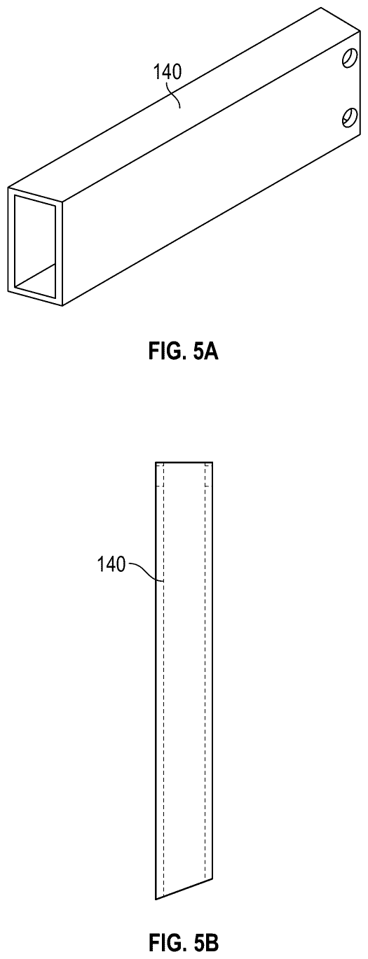

Referring now to FIGS. 1A-5B, the present invention features a dynamic foot brace device (100) for providing arch support to a foot of a user through energy storage at an ankle of the user during a step. In some embodiments, the device (100) may comprise a tension member (120) comprising a first end coupled to an arch of the foot and a second end coupled to the ankle. The tension member (120) may be configured to store energy from tension applied to the first end during a first phase of the step and release the energy to the foot as a propulsion force when the tension applied to the first end is released during a second phase of the step.

In some embodiments, the device (100) may further comprise a sleeve component (110) configured to fit the arch of the foot and the ankle. The first end of the tension member (120) may be coupled to the sleeve component (110) at the arch of the foot. The second end of the tension member (120) may be coupled to the sleeve component (110) at the ankle. In some embodiments, the device (100) may further comprise a tension member guide component (140) coupled to the sleeve component (110) such that the tension member (120) is disposed through the tension member guide component (140).

In some embodiments, the device (100) may further comprise an arch support component (130) disposed within the sleeve component (110) at the arch of the foot, configured to support the arch of the foot during the first phase of the step. In some embodiments, the arch support component (130) may comprise an arch plate. In some embodiments, the arch plate may comprise a rigid material (i.e. plastic, carbon fiber, thermoplastic, rigid polymer). In some embodiments, the arch plate is disposed on top of a shape-memory polymer cushion. In some embodiments, the arch support component (130) may comprise a flexible material (i.e. shape-memory polymer, nylon, leather, polyurethane, vinyl, polyester, nylon mesh).

In some embodiments, the arch support component (130) may comprise a strap assembly comprising a primary strap coupled to the first end of the tension member (120) and one or more secondary straps coupled to the sleeve component (110), configured to tighten against the arch of the foot in response to the tension applied to the first end of the tension member (120) during the first phase of the step. In some embodiments, a secondary strap of the one or more secondary straps may be coupled to the heel of the sleeve component (110). In some embodiments, a secondary strap of the one or more secondary straps may be coupled to the sleeve component (110) where the ball of the foot would go. In some embodiments, a secondary strap of the one or more secondary straps may be coupled to a toe of the sleeve component (110). In some embodiments, the primary strap and the one or more secondary straps may join at a junction. In some embodiments, the junction may be placed at the arch of the foot. In some embodiments, a material of each strap of the strap assembly may comprise an elastic material (i.e. carbon fiber, nylon, rubber, plastic, composite materials).

In some embodiments, the arch support component (130) may comprise a supporting layer coupled to the sleeve component (110) at the arch of the foot. In some embodiments, the supporting layer may be an additional layer of material on the inside of the sleeve component (110) at the arch of the foot, on the outside of the sleeve component (110) at the arch of the foot, or a combination thereof. In some embodiments, the supporting layer may comprise the same material as the sleeve component (110). In some embodiments, the supporting layer may comprise a rigid material (i.e. plastic, carbon fiber, thermoplastic, rigid polymer). In some embodiments, the supporting layer may comprise an elastic material (i.e. carbon fiber, rubber, shape-memory polymer, nylon, leather, polyurethane, vinyl, polyester, nylon mesh).

In some embodiments, the tension member (120) may comprise an elastic strap comprising carbon fiber, nylon, rubber, or a combination thereof, or a spring comprising a metal material, an elastomeric material, or a combination thereof. In some embodiments, the sleeve component (110) may comprise a flexible wearable material (i.e. cotton, nylon). In some embodiments, the tension member guide component (140) may comprise a rigid material (i.e. plastic, metal), a flexible material (i.e. rubber, nylon, carbon fiber), or a combination thereof.

The present invention features a method for providing arch support to a foot of a user through energy storage at an ankle of the user during a step. In some embodiments, the method may comprise applying a dynamic foot brace device (100) to the foot of the user. The device (100) may comprise a sleeve component (110) configured to fit an arch of the foot and the ankle. The device (100) may further comprise a tension member (120) comprising a first end coupled to the arch of the foot and a second end coupled to the ankle. The method may further comprise storing, by the tension member (120), energy from tension applied to the first end of the tension member (120) during a first phase of the step, the first phase of the step comprising placing the foot on a surface and shifting weight of the user onto the foot. The method may further comprise releasing, by the tension member (120), the energy to the foot as a propulsion force when the tension applied to the first end is released during a second phase of the step, the second phase of the step comprising lifting the foot off of the surface.

In some embodiments, the device (100) may further comprise an arch support component (130) disposed within the sleeve component (110) at the arch of the foot, configured to support the arch of the foot during the first phase of the step. In some embodiments, the device (100) may further comprise a tension member guide component (140) coupled to the sleeve component (110) such that the tension member (120) is disposed through the tension member guide component (140). In some embodiments, the tension member (120) may comprise an elastic strap comprising carbon fiber, nylon, rubber, or a combination thereof, or a spring comprising a metal material, an elastomeric material, or a combination thereof.

In some embodiments, the arch support component (130) may comprise an arch plate. In some embodiments, the arch plate may comprise a rigid material (i.e. plastic, carbon fiber, thermoplastic, rigid polymer). In some embodiments, the arch plate is disposed on top of a shape-memory polymer cushion. In some embodiments, the arch support component (130) may comprise a flexible material (e.g. shape-memory polymer, nylon, leather, polyurethane, vinyl, polyester, nylon mesh).

In some embodiments, the arch support component (130) may comprise a strap assembly comprising a primary strap coupled to the first end of the tension member (120) and one or more secondary straps coupled to the sleeve component (110), configured to tighten against the arch of the foot in response to the tension applied to the first end of the tension member (120) during the first phase of the step. In some embodiments, a secondary strap of the one or more secondary straps may be coupled to the heel of the sleeve component (110). In some embodiments, a secondary strap of the one or more secondary straps may be coupled to the sleeve component (110) where the ball of the foot would go. In some embodiments, a secondary strap of the one or more secondary straps may be coupled to a toe of the sleeve component (110). In some embodiments, the primary strap and the one or more secondary straps may join at a junction. In some embodiments, the junction may be placed at the arch of the foot. In some embodiments, a material of each strap of the strap assembly may comprise an elastic material (i.e. carbon fiber, nylon, rubber, plastic, composite materials).

In some embodiments, the arch support component (130) may comprise a supporting layer coupled to the sleeve component (110) at the arch of the foot. In some embodiments, the supporting layer may be an additional layer of material on the inside of the sleeve component (110) at the arch of the foot, on the outside of the sleeve component (110) at the arch of the foot, or a combination thereof. In some embodiments, the supporting layer may comprise the same material as the sleeve component (110). In some embodiments, the supporting layer may comprise a rigid material (i.e. plastic, carbon fiber, thermoplastic, rigid polymer). In some embodiments, the supporting layer may comprise an elastic material (i.e. carbon fiber, rubber, shape-memory polymer, nylon, leather, polyurethane, vinyl, polyester, nylon mesh).

In some embodiments, the tension member (120) may comprise an elastic strap comprising carbon fiber, nylon, rubber, or a combination thereof, or a spring comprising a metal material, an elastomeric material, or a combination thereof. In some embodiments, the sleeve component (110) may comprise a flexible wearable material (i.e. cotton, nylon). In some embodiments, the tension member guide component (140) may comprise a rigid material (i.e. plastic, metal), a flexible material (i.e. rubber, nylon, carbon fiber), or a combination thereof.

The present invention features a dynamic foot brace device (100) for providing arch support to a foot of a user through energy storage at an ankle of the user during a step. In some embodiments, the device (100) may comprise a sleeve component (110) configured to fit an arch of the foot and the ankle. The device (100) may further comprise an arch support component (130) disposed within the sleeve component (110) at the arch of the foot, configured to support the arch of the foot during a first phase of the step. The device (100) may further comprise a tension member (120) having a first end and a second end. The first end may be coupled to the arch support component (130). The second end may be coupled to the sleeve component (110) at the ankle. The tension member (120) may be configured to store energy from tension applied to the first end during the first phase of the step and release the energy to the foot as a propulsion force when the tension applied to the first end is released during a second phase of the step. The device (100) may further comprise a tension member guide component (140) coupled to the sleeve component (110) such that the tension member (120) is disposed through the tension member guide component (140).

In some embodiments, the arch support component (130) may comprise an arch plate. In some embodiments, the arch plate may comprise a rigid material (i.e. plastic, carbon fiber, thermoplastic, rigid polymer). In some embodiments, the arch plate is disposed on top of a shape-memory polymer cushion. In some embodiments, the arch support component (130) may comprise a flexible material (e.g. shape-memory polymer, nylon, leather, polyurethane, vinyl, polyester, nylon mesh).

In some embodiments, the arch support component (130) may comprise a strap assembly comprising a primary strap coupled to the first end of the tension member (120) and one or more secondary straps coupled to the sleeve component (110), configured to tighten against the arch of the foot in response to the tension applied to the first end of the tension member (120) during the first phase of the step. In some embodiments, a secondary strap of the one or more secondary straps may be coupled to the heel of the sleeve component (110). In some embodiments, a secondary strap of the one or more secondary straps may be coupled to the sleeve component (110) where the ball of the foot would go. In some embodiments, a secondary strap of the one or more secondary straps may be coupled to a toe of the sleeve component (110). In some embodiments, the primary strap and the one or more secondary straps may join at a junction. In some embodiments, the junction may be placed at the arch of the foot. In some embodiments, a material of each strap of the strap assembly may comprise an elastic material (i.e. carbon fiber, nylon, rubber, plastic, composite materials).

In some embodiments, the arch support component (130) may comprise a supporting layer coupled to the sleeve component (110) at the arch of the foot. In some embodiments, the supporting layer may be an additional layer of material on the inside of the sleeve component (110) at the arch of the foot, on the outside of the sleeve component (110) at the arch of the foot, or a combination thereof. In some embodiments, the supporting layer may comprise the same material as the sleeve component (110). In some embodiments, the supporting layer may comprise a rigid material (i.e. plastic, carbon fiber, thermoplastic, rigid polymer). In some embodiments, the supporting layer may comprise an elastic material (i.e. carbon fiber, rubber, shape-memory polymer, nylon, leather, polyurethane, vinyl, polyester, nylon mesh).

In some embodiments, the tension member (120) may comprise an elastic strap comprising carbon fiber, nylon, rubber, or a combination thereof, or a spring comprising a metal material, an elastomeric material, or a combination thereof. In some embodiments, the sleeve component (110) may comprise a flexible wearable material (i.e. cotton, nylon). In some embodiments, the tension member guide component (140) may comprise a rigid material (i.e. plastic, metal), a flexible material (i.e. rubber, nylon, carbon fiber), or a combination thereof.

The present invention features a dynamic foot brace device (100) for providing arch support to a foot of a user through energy storage at an ankle of the user. In some embodiments, the device (100) may comprise a sleeve component (110) configured to fit an arch of the foot and the ankle. The device (100) may further comprise a tension member (120) having a first end and a second end. The first end may be coupled to the sleeve component (110) at the arch of the foot. The second end may be coupled to the sleeve component (110) at the ankle. In some embodiments, the tension member (120) may be configured to store energy from tension applied to the first end and release the energy to the foot as a propulsion force when the tension applied to the first end is released.

In some embodiments, the device (100) may further comprise a tension member guide component (140) coupled to the sleeve component (110) such that the tension member (120) is disposed through the tension member guide component (140). In some embodiments, the device (100) may further comprise an arch support component (130) disposed within the sleeve at the arch of the foot, shaped to match a shape of a non-collapsed arch, the arch support component (130) comprising a shape-memory polymer cushion and an arch plate disposed on top of the shape-memory polymer cushion. In some embodiments, the first end of the tension member (120) may be coupled to the arch support component (130).

In some embodiments, the tension member (120) may comprise an elastomeric spring. In some embodiments, the tension member (120) may comprise a strap. In some embodiments, the strap may comprise carbon fiber, nylon, rubber, or a combination thereof.

In some embodiments, the device (100) may be configured to be worn with sneakers, slides, or any shoe. In some embodiments, the device (100) may be configured to cover the user's toes, heel, or a combination thereof. In some embodiments, the device (100) may be configured to cover the entire foot and ankle. In some embodiments, the device (100) may be configured to cover the entire foot and ankle except for the toes, the heel, or a combination thereof. In some embodiments, the first end of the tension member (120) may be attached to the bottom of the arch support component (130). In some embodiments, the second end of the tension member (120) may be coupled to a side of the ankle just above the talus bone.

In some embodiments, the tension member (120) may comprise a plurality of member segments. In some embodiments, the tension member (120) may comprise a housing comprising one or more clamps for connecting the plurality of member segments together. In some embodiments, the one or more clamps may comprise aluminum. In some embodiments, the housing may comprise polytetrafluoroethylene tubing. In some embodiments, the tension member guide component (140) may comprise polytetrafluoroethylene tubing. In some embodiments, the shape-memory polymer cushion of the arch support component (130) may comprise polytetrafluoroethylene, polylactide, ethylene-vinyl acetate, any other material capable of exhibiting a shape-memory effect, or a combination thereof.

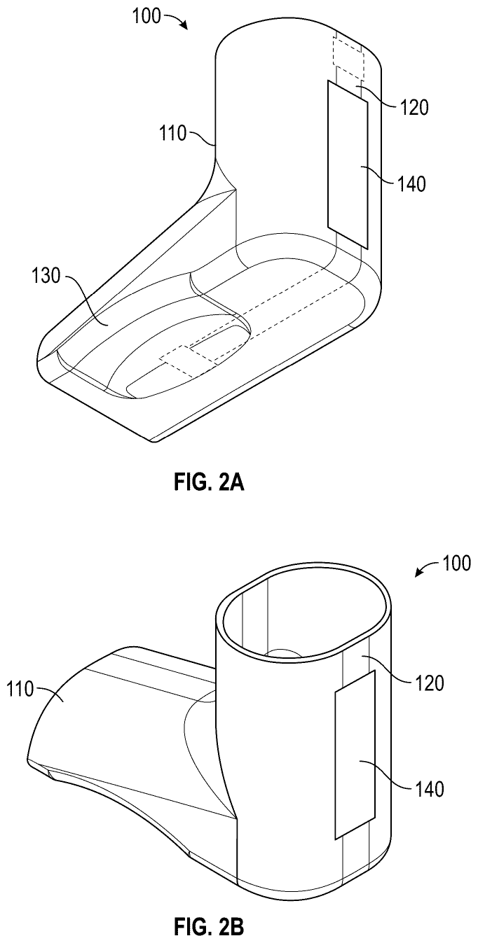

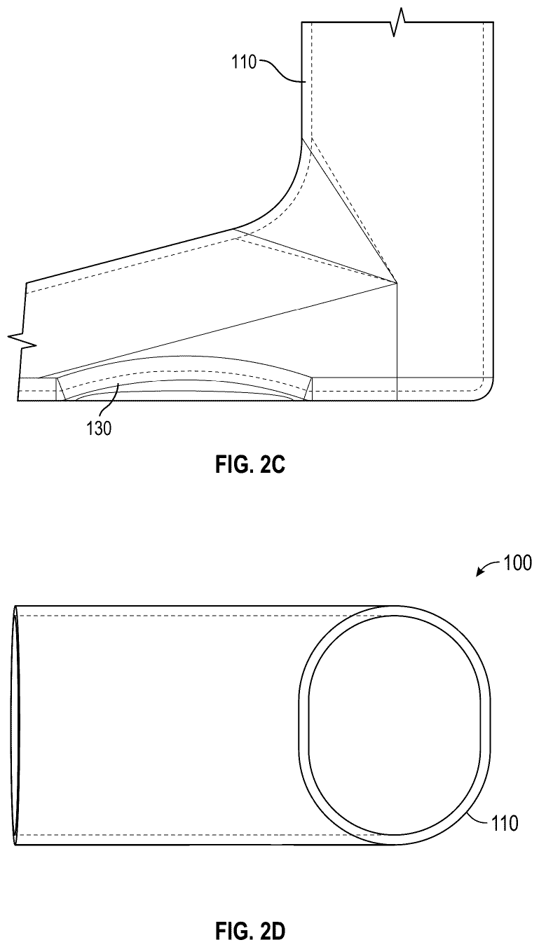

In some embodiments, the tension member (120) may be coupled to the ankle section of the sleeve and the arch section of the sleeve by stitches, adhesives, buckles, zippers, buttons, any component or mechanism for attaching two components together, or a combination thereof. This is depicted in FIG. 2A, examples of attachment points are depicted as shaded dotted boxes.

### EXAMPLE

The following is a non-limiting example of the present invention. It is to be understood that said example is not intended to limit the present invention in any way. Equivalents or substitutes are within the scope of the present invention.

A user suffers from a flat foot deformity that causes the arch of their foot to be misshapen. This causes discomfort while walking and applying pressure to the affected foot. The user applies the dynamic foot brace device to their foot and aligns the arch support component with the arch of their foot. The sleeve component of the device is pulled up such that the second end of the tension member aligns with the ankle. The user takes a step and shifts their weight onto the foot with the applied device. Their arch presses against the arch support component such that the shape of the arch is corrected. Simultaneously, the weight applied to the arch applies tension to the tension member that is stored there as energy. The user then lifts their foot and shifts their weight onto the other foot. The energy stored in the tension member is released as a propulsion force that drives their foot forward and helps them to complete their walking motion. This process is continued with every step throughout the walking process. This greatly aids the user, allowing them to walk more efficiently and with less pain.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A dynamic foot brace device (100) for providing arch support to a foot of a user through energy storage at an ankle of the user during a step, the device (100) comprising a tension member (120) comprising a first end coupled to an arch of the foot and a second end coupled to the ankle, configured to store energy from tension applied to the first end during a first phase of the step and release the energy to the foot as a propulsion force when the tension applied to the first end is released during a second phase of the step.

2. The device (100) of claim 1 further comprising a sleeve component (110) configured to fit the arch of the foot and the ankle, wherein the first end of the tension member (120) is coupled to the sleeve component (110) at the arch of the foot, wherein the second end of the tension member (120) is coupled to the sleeve component (110) at the ankle.

3. The device (100) of claim 2 further comprising a tension member guide component (140) coupled to the sleeve component (110) such that the tension member (120) is disposed through the tension member guide component (140).

4. The device (100) of claim 2 further comprising an arch support component (130) disposed within the sleeve component (110) at the arch of the foot, configured to support the arch of the foot during the first phase of the step.

5. The device (100) of claim 4, wherein the arch support component (130) comprises an arch plate.

6. The device (100) of claim 5, wherein the arch plate comprises a rigid material.

7. The device (100) of claim 5, wherein the arch plate is disposed on top of a shape-memory polymer cushion.

8. The device (100) of claim 4, wherein the arch support component (130) comprises a strap assembly comprising a primary strap coupled to the first end of the tension member (120) and one or more secondary straps coupled to the sleeve component (110), configured to tighten against the arch of the foot in response to the tension applied to the first end of the tension member (120) during the first phase of the step.

9. The device (100) of claim 4, wherein the arch support component (130) comprises a supporting layer coupled to the sleeve component (110) at the arch of the foot.

10. The device (100) of claim 1, wherein the tension member (120) comprises an elastic strap comprising carbon fiber, nylon, rubber, or a combination thereof, or a spring comprising a metal material, an elastomeric material, or a combination thereof.

11. A method for providing arch support to a foot of a user through energy storage at an ankle of the user during a step, the method comprising:
a. applying a dynamic foot brace device (100) to the foot of the user, the device (100) comprising:
i. a sleeve component (110) configured to fit an arch of the foot and the ankle; and
ii. a tension member (120) comprising a first end coupled to the arch of the foot and a second end coupled to the ankle;
b. storing, by the tension member (120), energy from tension applied to the first end of the tension member (120) during a first phase of the step, the first phase of the step comprising placing the foot on a surface and shifting weight of the user onto the foot; and
c. releasing, by the tension member (120), the energy to the foot as a propulsion force when the tension applied to the first end is released during a second phase of the step, the second phase of the step comprising lifting the foot off of the surface.

12. The method of claim 11, wherein the device (100) further comprises an arch support component (130) disposed within the sleeve component (110) at the arch of the foot, configured to support the arch of the foot during the first phase of the step.

13. The method of claim 11, wherein the device (100) further comprises a tension member guide component (140) coupled to the sleeve component (110) such that the tension member (120) is disposed through the tension member guide component (140).

14. The method of claim 11, wherein the tension member (120) comprises an elastic strap comprising carbon fiber, nylon, rubber, or a combination thereof, or a spring comprising a metal material, an elastomeric material, or a combination thereof.

15. A dynamic foot brace device (100) for providing arch support to a foot of a user through energy storage at an ankle of the user during a step, the device (100) comprising:
a. a sleeve component (110) configured to fit an arch of the foot and the ankle;
b. an arch support component (130) disposed within the sleeve component (110) at the arch of the foot, configured to support the arch of the foot during a first phase of the step;
c. a tension member (120) having a first end and a second end, wherein the first end is coupled to the arch support component (130), wherein the second end is coupled to the sleeve component (110) at the ankle, configured to store energy from tension applied to the first end during the first phase of the step and release the energy to the foot as a propulsion force when the tension applied to the first end is released during a second phase of the step; and
d. a tension member guide component (140) coupled to the sleeve component (110) such that the tension member (120) is disposed through the tension member guide component (140).

16. The device (100) of claim 15, wherein the arch support component (130) comprises an arch plate.

17. The device (100) of claim 16, wherein the arch plate comprises a rigid material.

18. The device (100) of claim 16, wherein the arch plate is disposed on top of a shape-memory polymer cushion.

19. The device (100) of claim 15, wherein the arch support component (130) comprises a strap assembly comprising a primary strap coupled to the first end of the tension member (120) and one or more secondary straps coupled to the sleeve component (110), configured to tighten against the arch of the foot in response to the tension applied to the first end of the tension member (120) during the first phase of the step.

20. The device (100) of claim 15, wherein the arch support component (130) comprises a supporting layer coupled to the sleeve component (110) at the arch of the foot.
